(19) United States
(12) Patent Application Publication
(10) Pub. No.: US 2025/0143639 A1
(43) Pub. Date: May 8, 2025

(54) MODULATING USER BEHAVIOR AND BIOFEEDBACK USING A NEUROIMAGING AND NEUROSTIMULATING SYSTEM

(71) Applicant: NIReality LLC, Richmond, VA (US)

(72) Inventors: Ryan Casey BOUTWELL, Richmond, VA (US); Jason Michael STROHMAIER, West Harwich, MA (US); Thibault Pierre Thierry ROUMENGOUS DE FESTES, Richmond, VA (US); Daniel Collins Rodenhaver, Richmond, VA (US)

(73) Assignee: NIReality LLC, Richmond, VA (US)

(21) Appl. No.: 19/017,011

(22) Filed: Jan. 10, 2025

Related U.S. Application Data
(63) Continuation-in-part of application No. PCT/US2023/069769, filed on Jul. 7, 2023.
(60) Provisional application No. 63/389,282, filed on Jul. 14, 2022.

(51) Int. Cl. A61B 5/00; A61B 5/16; G16H 40/67; G16H 50/30

(52) U.S. Cl. CPC A61B 5/486; A61B 5/165; G16H 40/67; G16H 50/30

(57) ABSTRACT

A method for providing biofeedback to a user may include receiving a first signal associated with one or more biometric properties of a user, and determining a baseline cognitive state of the user based on the first signal. The method may include iteratively detecting an instant cognitive state of the user, and receiving, from the detecting step, a second signal associated with the one or more biometric properties. The method may include determining whether the second signal corresponds to threshold(s). Responsive to such determination, the method may include initiating first feedback action(s). Responsive to initiating the first feedback action(s), the method may include determining whether the instant cognitive state of the user is between the threshold(s) and the baseline cognitive state, and if not, initiating second feedback action(s).

### CROSS-REFERENCE TO RELATED APPLICATIONS

This application is a continuation-in-part (CIP) application of, and claims priority under 35 U.S.C. § 111(a) to, International Application No. PCT/US2023/069769, filed Jul. 7, 2023, which claims priority under 35 U.S.C. § 119 to U.S. Provisional Patent Application No. 63/389,282, filed Jul. 14, 2022, the entire contents of each of which are fully incorporated herein by reference.

### TECHNICAL FIELD

The present disclosure relates generally to modulating biofeedback behavior using a neuroimaging and neurostimulating system. In particular, the present disclosure relates to systems and methods for providing biofeedback to a user based on the user's adapting cognitive state.

### BACKGROUND

Near-infrared spectroscopy (NIRS) devices interrogate biological tissue using a selection of light wavelengths in the red and near-infrared (NIR) region of the electromagnetic spectrum. These wavelengths are particularly well suited for deep light penetration through tissue, versus lower wavelengths of light that are scattered or absorbed by confounding factors in the body and thus cannot reach the tissue depth of these red and NIR wavelengths. NIRS devices generally feature at least two wavelengths of light output in this range and at least one detector, and including additional optical elements can allow different depths of sensing.

Red and near-infrared wavelengths are particularly effective for non-invasively sensing different molecular states of hemoglobin in various body tissues. Unfortunately, existing NIRS devices are typically expensive, large desktop units with disintegrated sensor and processing systems. This lack of portability limits the usefulness of NIRS outside of the surgical suite, laboratory, and research environments. Some portable solutions include a sensor-only patch with wired communication to a separate portable, pocketable, or head-worn processing and communications unit. These changes represent only a nominal improvement, as the processing unit is itself not fully wearable and risks physically detaching from the sensor unit through movement or cable weight. These limitations greatly decrease the wearability and utility of such systems. These semi-ambulatory systems are also typically not designed to be used in parallel, where individual NIRS sensor systems work in tandem across the body or across a population to continually sense physiological features at multiple places using a common interface. Non-ambulatory systems can have more sensor inputs, but these are limited by the total number of ports designed into the physical system itself. Therefore, there exists a need for integrated NIRS systems and methods of using those systems to interrogate biological tissue.

Additionally, existing NIRS systems and methods are unable to be coupled with electrophysiological sensing capabilities in an integrated and/or wearable package. Most portable, wearable electrophysiological sensing systems, such as those used in electroencephalography (EEG), use a very small number of electrodes for cognitive sensing and do not have the additional benefit of optical hemodynamic sensing for additional contextual awareness.

Further, existing NIRS systems and methods are unable to provide adaptive biofeedback to a user based on the user's changing cognitive and/or physiological state.

### SUMMARY

In some embodiments, a method for providing biofeedback to a user may include receiving a first signal associated with one or more biometric properties of the user. The method may include determining a baseline cognitive state of the user based on the first signal. The method may include iteratively detecting an instant cognitive state of the user. The method may include receiving, from the detecting step, a second signal associated with the one or more biometric properties. The method may include determining whether the second signal corresponds to one or more thresholds. Responsive to determining that the second signal corresponds to the one or more thresholds, the method may include initiating one or more first feedback actions. Responsive to initiating the one or more first feedback actions, the method may include determining whether the instant cognitive state of the user is between the one or more thresholds and the baseline cognitive state. Responsive to determining the instant cognitive state of the user is not between the one or more thresholds and the baseline cognitive state, the method may include initiating one or more second feedback actions.

In some embodiments, the one or more biometric properties may include one or more optical biometric properties and one or more non-optical biometric properties.

In some embodiments, the one or more optical biometric properties may include one or more of tissue oxygenation, heart rate, respiratory rate, oxygen saturation, blood pressure, or combinations thereof. In some embodiments, the one or more non-optical biometric properties may include one or more of EEG, electrooculography (EOG), electromyography (EMG), bioimpedance circuitry, thermal properties, mechanical properties, or combinations thereof.

In some embodiments, the one or more thresholds may be based on one or more of a level of effort by the user in one or more tasks, a level of engagement of the user in the one or more tasks, a level of fatigue of the user in the one or more tasks, or combinations thereof.

In some embodiments, the one or more first and second feedback actions may include one or more of an optical biometric property reading, a non-optical biometric property reading, one or more types of stimulation, a content modification, or combinations thereof.

In some embodiments, the one or more types of stimulation may include one or more of transcranial direct current stimulation ((DCS), transcranial alternating current stimulation (tACS), vagus nerve stimulation (nVNS), transcutaneous electrical nerve stimulation (TENS), transcranial magnetic stimulation (TMS), sound stimulation, optical stimulation, mechanical stimulation, or combinations thereof.

In some embodiments, the content modification may include one or more of a change in task type, a change in task difficulty, a change in training, a change in medical treatment, a delivery of one or more types of therapy, a connection to an artificial intelligence (AI) system, or combinations thereof.

In some embodiments, the one or more types of therapy may include one or more of cognitive therapy, psychological therapy, physical therapy, or combinations thereof.

In some embodiments, the one or more first and second feedback actions may be provided to a user via an environmental property. The environmental property may include one or more of temperature, pressure, chemical composition, sound, light, motion, or combinations thereof.

In some embodiments, a method for providing biofeedback to a user may include assigning a baseline cognitive state to the user based on one or more factors, the baseline cognitive state associated with one or more biometric properties. The method may include iteratively detecting an instant cognitive state of the user. The method may include receiving, from the detecting step, a first signal associated with the one or more biometric properties. The method may include determining whether the first signal corresponds to one or more thresholds. Responsive to determining that the first signal corresponds to the one or more thresholds, the method may include initiating one or more first feedback actions. Responsive to initiating the one or more first feedback actions, the method may include determining whether the instant cognitive state of the user is between the one or more thresholds and the baseline cognitive state. Responsive to determining the instant cognitive state of the user is not between the one or more thresholds and the baseline cognitive state, the method may include initiating one or more second feedback actions.

In some embodiments, the one or more factors may include one or more of age, sex, gender, education level, profession, medical history, content familiarity, task engagement, task effort, task performance, environmental factors, or combinations thereof.

In some embodiments, the one or more biometric properties may include one or more optical biometric properties and one or more non-optical biometric properties. In some embodiments, the one or more optical biometric properties may include one or more of tissue oxygenation, heart rate, respiratory rate, oxygen saturation, blood pressure, or combinations thereof. In some embodiments, the one or more non-optical biometric properties may include one or more of EEG, EOG, EMG, bioimpedance circuitry, thermal properties, mechanical properties, or combinations thereof.

In some embodiments, the one or more thresholds may be based on one or more of a level of effort by the user in one or more tasks, a level of engagement of the user in the one or more tasks, a level of fatigue of the user in the one or more tasks, or combinations thereof.

In some embodiments, the one or more first and second feedback actions may include one or more of an optical biometric property reading, a non-optical biometric property reading, one or more types of stimulation, a content modification, or combinations thereof.

In some embodiments, the content modification may include one or more of a change in task type, a change in task difficulty, a change in virtual training, a change in medical treatment, a delivery of one or more types of therapy, a connection to an Al system, or combinations thereof.

In some embodiments, a method for providing biofeedback to a plurality of users may include receiving a respective first signal associated with one or more biometric properties of each user of the plurality of users. The method may include iteratively detecting a respective instant cognitive state of each user. The method may include receiving, from the detecting step, a respective second signal associated with the one or more biometric properties of at least a first user of the plurality of users. The method may include determining whether the respective second signal corresponds to one or more thresholds. Responsive to determining that the respective second signal corresponds to the one or more thresholds, the method may include initiating one or more first feedback actions associated with at least a second user of the plurality of users. Responsive to initiating the one or more first feedback actions, the method may include determining whether the respective instant cognitive state of each user is between the one or more thresholds and a respective baseline cognitive state of each user. Responsive to determining the respective instant cognitive state of each user is not between the one or more thresholds and the respective baseline cognitive state, the method may include initiating, by the one or more processors, one or more second feedback actions associated with at least a third user of the plurality of users.

In some embodiments, the one or more biometric properties may include one or more optical biometric properties and one or more non-optical biometric properties. In some embodiments, the one or more optical biometric properties may include one or more of tissue oxygenation, heart rate, respiratory rate, oxygen saturation, blood pressure, or combinations thereof. In some embodiments, the one or more non-optical biometric properties may include one or more of EEG, EOG, EMG, bioimpedance circuitry, thermal properties, mechanical properties, or combinations thereof.

In some embodiments, the one or more thresholds may be based on one or more of a level of effort by each user in one or more tasks, a level of engagement of each user in the one or more tasks, a level of fatigue of each user in the one or more tasks, or combinations thereof.

In some embodiments, the one or more respective first and second feedback actions may include one or more of an optical biometric property reading, a non-optical biometric property reading, one or more types of stimulation, a content modification, or combinations thereof.

In some embodiments, at least two of the first, second, and third users may be the same.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1A depicts a top view of an embodiment of a substrate of a system for detecting biometric and neurophysiological parameters, in accordance with the present disclosure.

FIG. 1B depicts a side view of the embodiment of FIG. 1A of the substrate of a system for detecting biometric and neurophysiological parameters, in accordance with the present disclosure.

FIG. 1C depicts a bottom view of the embodiment of the substrate of a system for detecting biometric and neurophysiological parameters, in accordance with the present disclosure.

FIG. 1D depicts an embodiment of the substrate of a system for detecting biometric and neurophysiological parameters, in accordance with the present disclosure.

FIG. 2 depicts an embodiment of a substrate of a system for detecting biometric and neurophysiological parameters, in accordance with the present disclosure.

FIG. 3 depicts an embodiment of an electronics module of a system for detecting biometric and neurophysiological parameters, in accordance with the present disclosure.

FIG. 4 depicts an embodiment of a system for detecting biometric and neurophysiological parameters, in accordance with the present disclosure.

FIG. 5 is a flowchart of a method for detecting biometric and neurophysiological parameters, in accordance with the present disclosure.

FIG. 6 is a flowchart of a method for providing biofeedback to a user, in accordance with the present disclosure.

FIG. 7 is a flowchart of a method for providing biofeedback to a user, in accordance with the present disclosure.

FIG. 8 is a flowchart of a method for providing biofeedback to a plurality of users, in accordance with the present disclosure.

FIG. 9 depicts a system for detecting biometric and neurophysiological parameters, in accordance with the present disclosure.

### DETAILED DESCRIPTION

This disclosure is not limited to the particular systems, devices, and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. For example, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

It is to be understood that the mention of one or more method steps does not preclude the presence of additional method steps or intervening method steps between those steps expressly identified. It is also to be understood that the listing of one or more method steps in any order does not preclude the performance of such one or more methods steps in alternative orders, nor does it preclude the simultaneous performance of any such one or more method steps. Similarly, it is also to be understood that the mention of one or more components in a device or system does not preclude the presence of additional components or intervening components between those components expressly identified.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two components,” without other modifiers, means at least two components, or two or more components). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

Furthermore, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

Near-infrared spectroscopy devices interrogate biological tissue using a selection of light wavelengths in the red and NIR region of the electromagnetic spectrum. These wavelengths are particularly well suited for deep light penetration through tissue, versus lower wavelengths of light that are scattered or absorbed by confounding factors in the body and thus cannot reach the tissue depth of these red and NIR wavelengths. NIRS devices generally feature at minimum two wavelengths of light output in this range and at least one detector, and including additional optical elements can allow different depths of sensing.

Red and near-infrared wavelengths are particularly effective for non-invasively sensing different molecular states of hemoglobin in various body tissues. Hemoglobin is a strong absorber of light in the middle of the visible light spectrum but has a low optical extinction coefficient within the higher wavelengths of the visible range. Within the NIR wavelengths, for hemoglobin's oxygenation states, deoxy- and oxyhemoglobin's absorption spectra cross at an isosbestic point near 805 nm, allowing NIRS systems to differentiate oxygenation states of hemoglobin using light sources above and below this wavelength. With this differentiation, NIRS can be used for a variety of sensing mechanisms related to the body's circulatory and other functional systems.

Hemoglobin also allows for binding of ligands other than oxygen. These other molecular states of hemoglobin, such as carboxyhemoglobin and methemoglobin, have unique optical absorption characteristics in the NIR range. Investigating these molecular states can elucidate competitive binding and indicate histologic changes in tissue oxygenation such as tissue poisoning. Hemoglobin has a competitive binding efficiency for many molecules, such as carbon monoxide (CO), cyanide (CN-), sulfur monoxide (SO), sulfide (S2-), and others in these groups. Nitric oxide (NO) also binds to hemoglobin and can be detected optically. Investigating the NIR spectra of these additional bound states of hemoglobin can indicate tissue status and toxicity by inhibiting oxygen binding as well as enable sophisticated physiological monitoring of body systems.

NIRS systems may calculate oxygenation levels using the modified Beer-Lambert law (mBLL), which only requires one bank of light sources. Using the mBLL offers the translation of raw optical signals into actionable oxygenation details. Alternatively, NIRS systems may employ spatially resolved spectroscopy (SRS), which can use both short-and long-distance measurements. Separately, short channel information can be subtracted from long channel information to more accurately isolate, for example, brain activity and the contributions from internal (e.g., cerebral) vasculature and external (e.g., skin) vasculature.

Unfortunately, existing NIRS devices are typically expensive, large desktop units with disintegrated sensor and processing systems. This lack of portability limits the usefulness of NIRS outside of the surgical suite, laboratory, and research environments. Even in such controlled environments, these devices sometimes fail because they are difficult to integrate into a user's system when the planned testing involves any form of motion.

Some portable solutions include a sensor-only patch with wired communication to a separate portable, pocketable, or head-worn processing and communications unit. These changes represent only a nominal improvement, as the processing unit is itself not fully wearable and risks physically detaching the sensor unit through movement or cable weight. These limitations greatly decrease the wearability and utility of such systems. These semi-ambulatory systems are also typically not designed to be used in parallel, where individual NIRS sensor systems work in tandem across the body or across a population to continually sense physiological features at multiple places using a common interface. Non-ambulatory systems can have more sensor inputs, but these are limited by the total number of ports designed into the physical system itself. Therefore, there exists a need for integrated and/or modular NIRS systems and methods of using those systems to interrogate biological tissue.

Additionally, existing NIRS systems and methods are unable to be coupled with electrophysiological sensing capabilities in an integrated, modular, and/or wearable package. Most portable, wearable electrophysiological sensing systems, such as those used in electroencephalography (EEG), use a very small number of electrodes for cognitive sensing and do not have the additional benefit of optical hemodynamic sensing for additional contextual awareness.

Further, existing NIRS systems and methods are unable to provide adaptive biofeedback to a user based on the user's changing cognitive state.

Accordingly, the systems and methods disclosed herein may provide integrated, modular, and/or wearable optical and non-optical biometric sensing capabilities, such as a combination of electrophysiological, functional NIRS (fNIRS), and photoplethysmography (PPG)-based sensing, providing additional physiological metrics such as heart rate, respiratory rate, oxygen saturation (SpO₂), and blood pressure-related data streams in a single wearable system. The electrophysiological sensing capabilities described herein may provide electrophysiological sensors such as those used in EEG electrodes, e.g., covering portions of the frontal cortex of the brain; EOG electrodes, e.g., monitoring eye movement; EMG, e.g., monitoring muscle activation; and/or bioimpedance circuitry, e.g., monitoring tissue composition. In some embodiments, the systems disclosed herein may be modular such that different biometric and neurophysiological monitoring capabilities can be added or subtracted from the system to fit a user's specific requirements for system function, layout, size, etc. In some embodiments, the systems disclosed herein may be coupled with other biometric monitoring systems that may assess user aspects, such as bioimpedance, biochemical, thermal, and/or mechanical properties.

In some embodiments, the systems disclosed herein may provide electrical connections to a battery and an electronics module or modules that may process the electrophysiological signals and fNIRS signals separately or in tandem. The systems disclosed herein may be integrated into various types of equipment, uniforms, clothing, prosthetics, etc., for example, in an aircrew helmet at voids around the earcup, in other sections of the helmet interior, or at the nape of the neck just outside the helmet. The systems disclosed herein may also be worn as a standalone system, such as when a helmet is not worn, as in the above example, with the support electronics being integrated into, e.g., a skull cap or headband.

In some embodiments, the systems disclosed herein may provide a flexible, fan-shaped patch design that may conform to a portion of a user's body, such as the user's forehead, which may provide close coupling of the fNIRS optics and electrophysiological (e.g., EEG) electrodes to the skin, and may allow for more forehead movement and adaptation than a patch design covering the entire forehead. A major issue with EEG systems is the possibility of the electrodes lifting off from the body based on movement. The systems disclosed herein may include an adhesive having a large area to reduce the risk of electrode liftoff from the body in critical sensing locations. The systems disclosed herein may also use sweat-wicking materials in the adhesive itself which may ensure comfort over prolonged wear.

One issue with adhesive-based systems is ensuring that the design of the patch does not interfere with the hairline of the wearer, which can cause discomfort when the patch is removed. In instances where the user's forehead area is extremely small, the patch adhesive can be trimmed to ensure the patch does not touch the hairline without requiring the patch itself be cut.

In some embodiments, the systems disclosed herein may provide wings designed into the forehead patch that may allow the electrodes and fNIRS electronics to be set higher or lower on the forehead depending on the physical shape of the wearer's forehead. The wings themselves may be configured to maintain separation between the fNIRS electronics and EEG electrodes, which may decrease the potential for signal degradation to the EEG and EOG signals.

In some embodiments, the systems disclosed herein may provide a modular system whereby one or more components of the system (e.g., configured for monitoring or detecting certain properties of the user, and/or processing signals relating to those properties) can be integrated into a wearable device, such as a visor configured to be worn on a user's head. In some such embodiments where one or more of the above-mentioned components are integrated into a wearable device, other component(s) can be attached to other parts of the user's body such that the different components of the overall system can monitor the same and/or different properties of the user from different parts of the user's body (e.g., head, chest, etc.).

In some embodiments, the systems disclosed herein may be compatible with either commercial-off-the-shelf (COTS) wet silver/silver chloride (Ag/AgCl) electrodes or dry electrodes that may not require a gel/liquid interface to the skin for signal generation and quality. In some embodiments, the systems disclosed herein may provide an integrated fNIRS and EEG patch design having an adhesive area that augment the adhesive material on the COTS electrodes, which may reduce the risk of electrode separation from the forehead and may ensure longer wear duration.

Electrophysiological signals may be susceptible to electronic noise due to the low voltage nature of the raw signals themselves, which have to be sent over a significant physical distance before reaching the electronics module's frontend for filtering, amplification, and conversion. As such, in some embodiments, the systems disclosed herein may be designed to ensure that the electrophysiological signal lines (e.g., EEG) may be adequately protected from electronic noise through environmental factors and/or the fNIRS subsystem itself.

In some embodiments, the systems disclosed herein may be configured to have medical-grade elastomer encapsulation, which may allow for significant shielding integrated into the wearable substrate itself with the electrode lines running within the insulated/shielded material.

In some embodiments, the systems disclosed herein may provide electrode pad locations and cables to the electrodes that may also be locally shielded either within the elastomer or through use of a radio frequency (RF)-blocking, biocompatible combination adhesive. Shielding materials within the elastomer and/or adhesive can include thin copper sheets or other RF-blocking metallic or non-metallic material.

In some embodiments, the systems disclosed herein may provide a mold for the elastomer encapsulant that can be used to route the electrophysiological lines in a manner that may ensure that the pathway for the electrophysiological signals is separated as much as possible from the fNIRS subsystem to ensure local noise from that integrated patch area does not interfere with the electrophysiological signals. For example, while EOG signals may be less susceptible to interference than EEG signals, optimizing the cable routing within the mold may help to minimize degradation of the EOG signals. Similarly, for example, the routing for the prefrontal cortex groups (e.g., the measurement points at AF, F, and Fp using 10-20 EEG mapping) EEG lines can be molded away from the fNIRS subsystem so that signal interference remains low even if these areas are placed close together on the forehead.

In instances where the fNIRS electronics or optical signals may impact the electrophysiological (e.g., EEG) signals, or vice versa, the systems disclosed herein may be configured to adapt sampled data streams to toss out or vary filtering on either signal to account for the operation of the other subsystem. For example, if activation of the fNIRS optical electronics causes a noise spike in the electrophysiological readings, the milliseconds of data from the fNIRS subsystem firing sequence can be isolated, filtered, or discarded depending on the level and nature of the noise impacting the electrophysiological signal.

In some embodiments, the systems disclosed herein may be controlled by separate or integrated microcontroller(s) that may be configured to provide feedback to the user based on algorithms developed from the user's biometric property signals, such as oxygenation levels and electrical activity. In some embodiments, the systems disclosed herein may be combined with electrophysiological stimulation techniques such as tDCS, nVNS, TENS, and/or TMS, to stimulate the user based on situational need or perceived performance degradation. The systems and methods disclosed herein may thus be utilized in a variety of applications, such as flight environment, virtual reality, learning/training, teaming, gaming, human performance enhancement, rehabilitation acceleration, and others.

In some embodiments, the systems disclosed herein may provide electronics modules that can control sampling and data processing of the fNIRS, electrophysiological, and tDCS/nVNS subsystems. For example, the systems disclosed herein may be configured to communicate with an external system that can adapt its properties based on the data processed by the integrated fNIRS/electrophysiological system, and may trigger the tDCS/nVNS subsystem if certain criteria are met. Triggering of the tDCS/nVNS subsystem can be either local (e.g., on the electronics module) or remote (e.g., from the external communication system). In some embodiments, the electronics modules can be internal and/or external to a wearable device configured to house one or more components of the system, as further discussed below. For example, the electronics modules can be integrated into a wearable headset (e.g., a visor), and/or can be external to the wearable headset. In some embodiments, the electronics modules can communicate with other component(s) of the overall system via a wired and/or wireless (e.g., WiFi®) connection.

In some embodiments, the systems disclosed herein may allow details on the cognitive workload experienced by a user and identified by the patch system to be communicated with support personnel (e.g., in the flight environment) such that, for example, training and simulation activities may be adapted, and/or support personnel may be informed of any high burden scenarios experienced by a user (e.g., aircrew onboard a jet). In some embodiments, the use of NIRS-based hypoxia sensing can also alert, for example, aircrew and support personnel, of local or systemic failures in a user's breathing apparatus.

In some embodiments, the systems disclosed herein may allow for a user's cognitive workload data to be used to adapt training for the user based on the user's perception of task difficulty at a given time, such as in the virtual reality (VR) field. For example, if a performed task is either too difficult or too easy, the user may become disengaged from the task, while adequately difficult tasks may be likelier to maintain user engagement as they work or learn. By tracking workload levels over the short-or long-term, training or learning processes can be tailored to the capability or engagement level of the user, optimizing their progression.

In some embodiments, the systems disclosed herein may allow for tracking of user fatigue through perturbations of the NIRS and EEG signal that indicate reduced cognitive performance or inattention, while the EOG subsystem can identify changes such as drooping eyelids or changes in eye movement that could indicate the onset and/or depth of fatigue. Communications between this subsystem and the tDCS and/or nVNS subsystems can stimulate the user back into a state of increased alertness or, if these methods are insufficient, advise that the user cease activities until they have rested.

In some embodiments, the systems and methods disclosed herein may provide for the continuous monitoring of a user's physiological and cognitive state, and the adaptive providing of biofeedback to the user based on the user's changing physiological and cognitive state. The systems and methods disclosed herein may be configured to read a brain state of a user, or a plurality of users, and to quantify the user's effort and engagement in certain tasks by combining tracked thresholds and the expected difficulty of tasks managed by trainers, managers, system operators, etc. Based on this quantification, the systems and methods disclosed herein may be configured to provide content modification recommendations, e.g., a user should take a break from performing a task because the user has become over-stressed or fatigued.

In some embodiments, the systems and methods disclosed herein may be configured to provide neurostimulation techniques to help guide and entrain a user's brain toward a certain desired state (e.g., increased neuroplasticity) as determined by neuroimaging and by performance in a paired task (e.g., stimulus agnostic or stimulus integrated). The systems and methods disclosed herein may be configured to guide user behavior through continuous measuring of brain and physiological state to ascertain optimal performance outputs over fixed or variable periods of time. Guiding a user may involve, for example, changing a game difficulty during recreational gaming, adjusting learning behavior during virtual training, delivering therapy or medical treatment to a user, affecting a user's perception of time, etc.

In some embodiments, the systems and methods disclosed herein may be configured to change an environment of a user, such as by applying an indirect signal to adapt the user or an indirect change to a program and/or system based on the state of the user. Changing a user's environment may involve, for example, pairing external system(s) controlling light, sound, taste, touch, smell, etc., or other environmental stimuli (e.g., brightening lights in a room, reducing a room temperature, etc.).

In some embodiments, the systems and methods disclosed herein may be configured to provide a user with a partnered AI system based on the user's determined cognitive state. The use of an Al system may provide a user with a semi-human interaction perspective to guide the user toward long-term task effort, encouragement, focus, etc.

In some embodiments, the systems and methods disclosed herein may provide for the development of team performance metrics, such as a rate of cumulative team focus, decision making, effort, etc. The systems and methods disclosed herein may be configured to monitor changing cognitive and/or physiological states of multiple users simultaneously to provide feedback to one or more users of a group based on how each user handles individual tasks, a set of tasks, and/or their performance in certain tasks in comparison to other users. Based on continuous readings of team brain and physiological states, the systems and methods disclosed herein may provide for adapting of content to improve team performance, effort, etc., and help to prevent team fatigue, burn-out, etc.

Reference will now be made in detail to example embodiments of the disclosed technology that are illustrated in the accompanying drawings and disclosed herein. Wherever convenient, the same reference numbers will be used throughout the drawings to refer to the same or like parts.

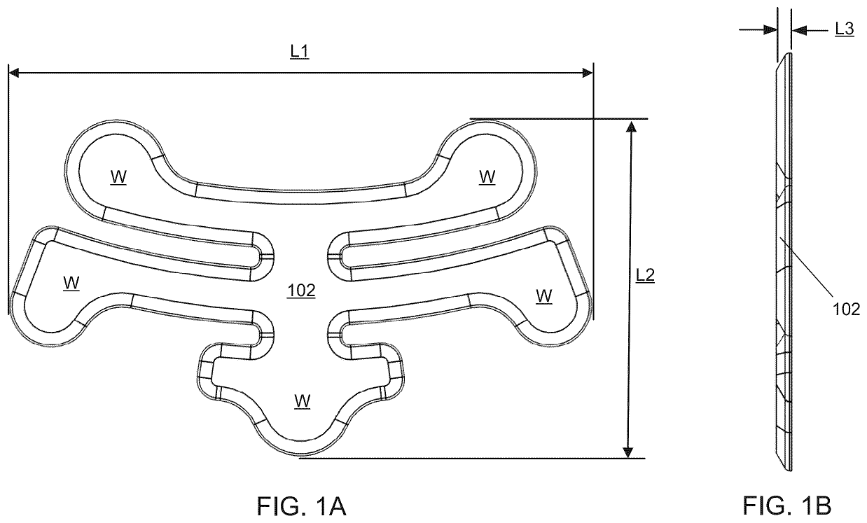

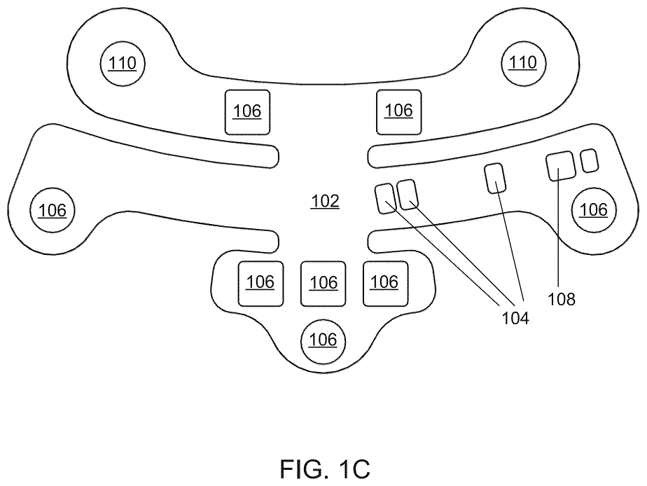

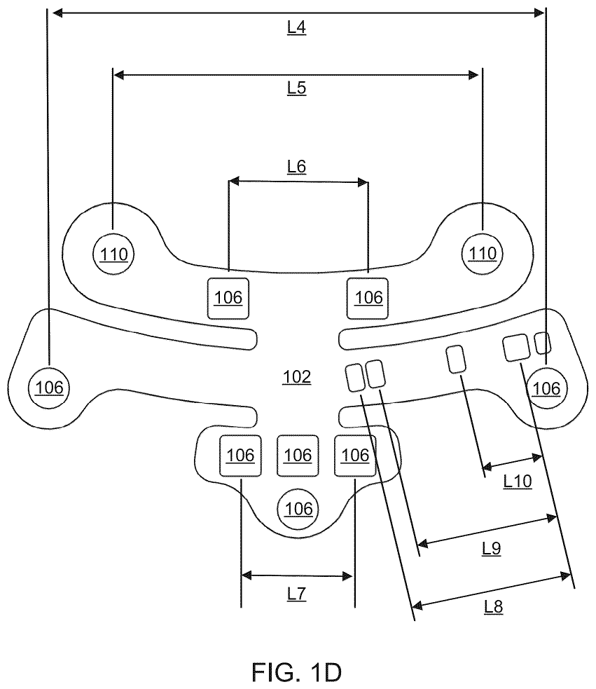

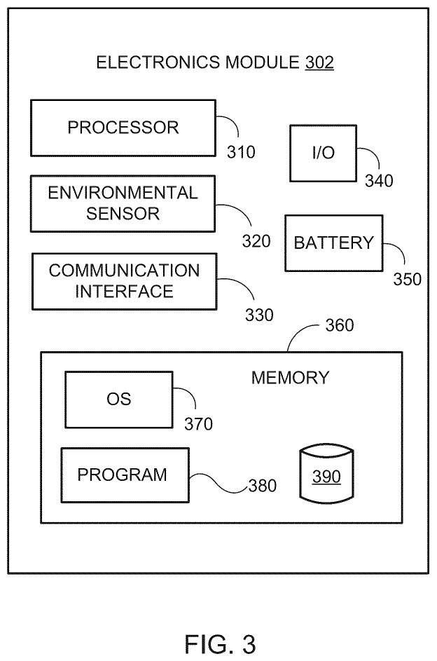

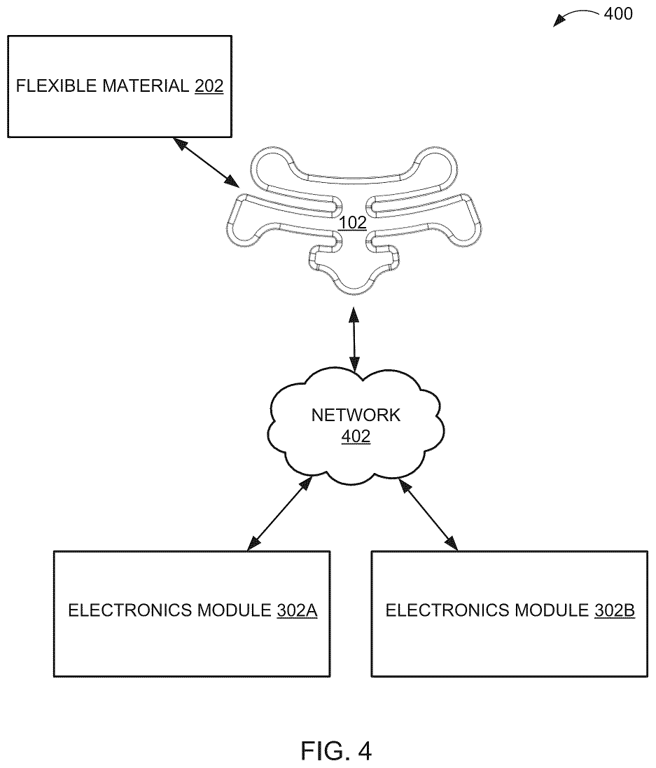

FIGS. 1A-ID and 2 depict embodiments of a substrate 102 for detecting biometric and neurophysiological parameters, in accordance with the present disclosure. The substrate 102 may be included in a system 400, along with a flexible material 202, a first electronics module (302A), and/or a second electronics module (302B), as further discussed below with respect to FIGS. 3 and 4.

In some embodiments, the substrate 102 may be coupled to a flexible material 202 (FIG. 4). For example, the substrate 102 may be configured to be placed on top of, or embedded within, a flexible material 202. As discussed herein, the flexible material 202 may improve the capability of substrate 102 to conform to a body part of a user, such as the user's forehead. The substrate 102 and/or the flexible material 202 may also provide for electrical insulation and/or optical isolation of any fNIRS and/or PPG optics, and/or any electrical connection lines to EEG electrodes mounted on substrate 102. The flexible material 202 may include one or more adhesive materials, such as sweat-resistant or sweat-wicking adhesives, such that the substrate 102 may removably adhere to a body part of the user. In some embodiments, these adhesives may provide unique optical, electrical, thermal, and/or mechanical properties that may be selected to meet sensor requirements for each area (e.g., user body part) covered by a sensor, as further discussed herein.

In some embodiments, the substrate 102 may include one or more materials, such as silicone, nylon, epoxy, a bioinert polymer, a biocompatible polymer, a woven or nonwoven textile, an adhesive film, a flexible circuit board, flexible sensors and electronics, or a combination thereof. In some embodiments, the substrate 102 and any components mounted onto the substrate may be configured to provide mechanical flexibility, allowing the substrate 102 to conform to and/or adhere to a surface. In some embodiments, the substrate 102 may be configured to be integrated into clothing or other equipment designed to be worn or applied to a user.

In some embodiments, the shape and/or dimensions of the substrate 102 may be different depending on the specific patient and/or use case. For example, as particularly shown in FIG. 1A, the substrate 102 may include a plurality of wings, W, that may allow the electrophysiological electrodes and fNIRS electronics to be set, for example, higher or lower on a user's forehead, and may provide separation between the electrophysiological electrodes and INIRS electronics to decrease potential signal degradation between the two, as discussed herein. Additionally, substrate 102 may also be configured of any size. For example, as shown in FIG. 1A, substrate 102 may include a length L1 of approximately 5.572 inches and a width L2 of approximately 3.221 inches. As shown in FIG. 1B, substrate 102 may include a thickness L3 of approximately 0.118 inches.

As shown in FIG. 1C, in some embodiments, the substrate 102 may include one or more first detectors 104 capable of detecting one or more optical biometric properties of a user. For example, the optical biometric properties may include tissue oxygenation, heart rate, respiratory rate, oxygen saturation, blood pressure, and the like. In some embodiments, the first detectors 104 may include optical detectors configured to detect specific sets of wavelengths emitted from light source 108, as further discussed below. The optical detectors may be configured to detect backscattered light from light source 108, as the backscattered light travels through tissue. In some embodiments, the optical detectors may comprise a single optical detector. In other embodiments, the optical detectors may comprise multiple optical detectors, such as 2 optical detectors, 3 optical detectors, 4 optical detectors, 5 optical detectors, and so on. In some embodiments, the optical detectors may be capable of detecting the first set of wavelengths, as described herein. In some embodiments, the optical detectors may be capable of detecting the first set of wavelengths and the second set of wavelengths, as described herein.

In some embodiments, the substrate 102 may include one or more second detectors 106 capable of detecting one or more non-optical biometric properties of a user. For example, the non-optical biometric properties may include electrophysiological properties, such as EEG, EOG, EMG, bioimpedance circuitry, thermal properties, mechanical properties, and the like. As discussed herein, providing a substrate 102 that may detect both optical and non-optical properties of a user may enable the user to be continuously monitored in certain environments (e.g., in the flight environment). In some embodiments, as further discussed herein, this continuous monitoring may enable as-required stimulation of the user, for example, via the substrate 102 or another component of system 400 external to the substrate 102.

First detectors 104 and second detectors 106 may be mounted to substrate 102 in a variety of positions. For example, as particularly shown in FIGS. 1C-1D, first detectors 104 may be attached to one of the wings, W, of the substrate 102, and at one or more distances from a light source 108 also attached to the substrate 102. For example, first detectors 104 may be placed on the substrate 102 at distances of L8, L9, and L10 from the light source 108, where L8 may be approximately 1.575 inches, L9 may be approximately 1.378 inches, and L10 may be approximately 0.591 inches.

Second detectors 106 may be attached to one or more wings, W, of the substrate 102, and at one or more distances from one another, first detectors 104, and/or terminals 110 (further discussed below). For example, second detectors 106 may be placed on opposite wings of substrate 102 and at distances of L4 and L6 from each other, where L4 may be approximately 4.785 inches, and L6 may be approximately 1.339 inches. As another example, second detectors 106 may be placed on a single wing, W, at a distance of L7 from each other, where L7 is approximately 1.102 inches.

In some embodiments, the placement of the second detectors 106 on the substrate 102 may be based on the international 10-20 electrode system with focus on the prefrontal cortex (e.g., Fp, AF, and F groups). In some embodiments, second detectors 106 may include EEG electrodes that may be expandable from a single channel to multi-channel EEG. In some embodiments, the second detectors 106 may include EOG electrodes that may be placed around a user's eyes, for example to track eye movement. In some embodiments, the second detectors 106 may include EMG electrodes to track real-time muscle activation and/or metabolic demand.

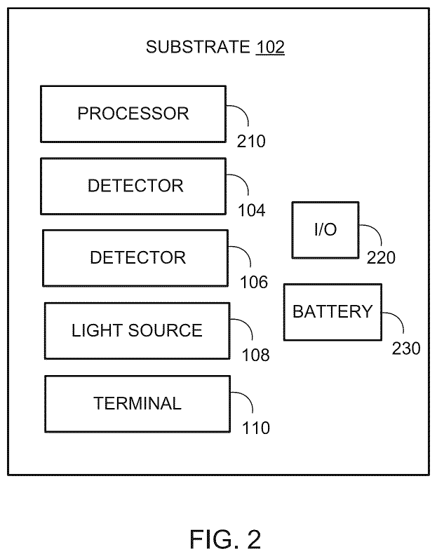

As shown in FIG. 2, the substrate 102 may further include a processor 210, an input/output (I/O) device 220, and a power source 230 (e.g., a battery) configured to power the substrate 102.

The I/O device 220 may be configured to connect the substrate 102 to one or more other components of system 400 or one or more components external to system 400, such as a computing device (e.g., a laptop or other “smart” device).

The light source 108 may include a single light source. In other embodiments, the light source 108 may include multiple light sources, such as 2 light sources, 3 light sources, 4 light sources, 5 light sources, and so on. In some embodiments, each light source may include one or more light emitting diodes (LEDs). In some embodiments, each light source may include a single tunable light source such as a broadband LED coupled with a miniature monochromator. In some embodiments, each light source may include one or more laser diodes. In an embodiment, the light source 108 may include a light source driver capable of selecting between the different light sources or selecting the wavelength from a tunable light source.

In some embodiments, the light source 108 may be capable of emitting a first set of wavelengths of red or near-infrared light. In some embodiments, each light source within the light source 108 may be capable of independently emitting a wavelength. The first set of wavelengths may comprise 1 wavelength, 2 wavelengths, 3 wavelengths, 4 wavelengths, 5 wavelengths, 6wavelengths, 7 wavelengths, 8 wavelengths, 9 wavelengths, 10 wavelengths, or any other number of wavelengths known in the art. In some embodiments, each wavelength within the first set of wavelengths may independently be from about 650 nm to about 950 nm. Each wavelength may be, for example, about 650 nm, about 655 nm, about 660 nm, about 665 nm, about 670 nm, about 675 nm, about 680 nm, about 685 nm, about 690 nm, about 695 nm, about 700 nm, about 705 nm, about 710 nm, about 715 nm, about 720 nm, about 725 nm, about 730 nm, about 735 nm, about 740 nm, about 745 nm, about 750 nm, about 755 nm, about 760 nm, about 765 nm, about 770 nm, about 775 nm, about 780 nm, about 785 nm, about 790 nm, about 795 nm, about 800 nm, about 805 nm, about 810 nm, about 815 nm, about 820 nm, about 825 nm, about 830 nm, about 835 nm, about 840 nm, about 845 nm, about 850 nm, about 855 nm, about 860 nm, about 865 nm, about 870 nm, about 875 nm, about 880 nm, about 885 nm, about 890 nm, about 895 nm, about 900 nm, about 905 nm, about 910 nm, about 915 nm, about 920 nm, about 925 nm, about 930 nm, about 935 nm, about 940 nm, about 945 nm, about 950 nm, or any range between any two of these values, including endpoints. In some embodiments, each wavelength within the first set of wavelengths may be greater than about 805 nm. In some embodiments, the average of the first set of wavelengths may be greater than about 805 nm. In certain embodiments, the first set of wavelengths may include five individual wavelengths to interrogate the targeted tissue: one in the red region below 730 nm, one in the NIR region below the 805 nm isosbestic point, one near or at the 805 nm isosbestic point, and two in the NIR region above the isosbestic point.

Turning to FIGS. 3 and 4, system 400 may include one or more electronics modules 302, e.g., 302A and 302B. Each electronics module 302A, 302B may be configured with the same or similar components; however, in some embodiments, electronics module 302A and 302B may be configured to detect and process different types of parameters of a user. For example, electronics module 302A may be configured to detect and process optical biometric properties of a user, while electronics module 302B may be configured to detect and process non-optical biometric properties of a user. In some embodiments, system 400 may include a single electronics module 302 that may include the hardware and functionality of electronics modules 302A and 302B, as discussed herein, and may be configured to carry out the program instructions for both electronics modules 302A and 302B, as discussed herein, within a single, integrated package.

In some embodiments, electronics module 302A may be communicatively coupled to the substrate 102, and may include a processor 310, a memory device 360, and instructions stored on the memory device 360 that may direct the processor 310 to perform one or more actions. The electronics module 302A may be configured to receive a signal from the one or more first detectors 104 mounted on the substrate 102, and process the received signal based on one or more first predefined algorithms. Based on the processed signal, the electronics module 302A may be configured to determine whether the user, e.g., on or to which substrate 102 is coupled, requires one or more first feedback actions. As used herein, the term “communicatively coupled” means configured to communicate and/or connect via one or more methods of communication, such as a wireless connection (e.g., WiFi®), a wired connection, a Bluetooth connection, a near-field communication (NFC) connection, a radio frequency identification (RFID) connection, etc.

In some embodiments, electronics module 302B may also be communicatively coupled to the substrate 102 and may include the same or similar components to that of electronics module 302A. Electronics module 302B may, however, be configured to receive a signal from the one or more second detectors 106 mounted on the substrate 102, and process the received signal based on one or more second predefined algorithms. Based on the processed signal, the electronics module 302B may be configured to determine whether the user, e.g., on or to which substrate 102 is coupled, requires one or more second feedback actions.

In some embodiments, at least one of the electronics modules 302A, 302B may be configured to determine that the user either requires no feedback actions, requires at least one of the first feedback actions, or requires at least one of the second feedback actions.

In some embodiments, the first and/or second feedback actions may include having an additional optical biometric property reading taken, having an additional non-optical biometric property reading taken, initiating one or more types of stimulation to be communicated to the user, and/or communicating with an external processor to enact a change in system behavior (e.g., biometrics of interest, operating mode, conditions of user stimulation, etc.).

In some embodiments, the first and/or second feedback actions may be provided to the user through changes in the user's real environment or virtual environment, e.g., via a VR device. In some embodiments, the first and/or second feedback actions may be perceived by the user, not perceived by the user, or partially perceived by the user. In some embodiments, the first and/or second feedback actions may be provided to the user pharmacologically, e.g., through intravascular, intramuscular, intraosseous, and/or other routes. In some embodiments, the first and/or second feedback actions may be provided to the user via some type of external environmental property, such as through a change in temperature, pressure, chemical composition, sound (e.g., continuous sound waves with changing pitch), light, motion, smell, feel, taste, and the like.

In some embodiments, the types of stimulation may be provided to the user via one or more terminals 110 mounted on the substrate 102 (FIGS. 1C-1D). In some embodiments, the types of stimulation may include tDCS, tACS, nVNS, TENS, TMS, ultrasound stimulation, optical stimulation, mechanical stimulation, or other types of invasive or non-invasive stimulation.

As shown in FIG. 3, the electronics module 302 may further include one or more environmental sensors 320, a communication interface 330, an I/O device 340, and an energy storage device 350 (e.g., a battery) configured to power the electronics module 302. Memory device 360 may include an operating system (OS) 370 and program 380, and a database 390. Operating system 370 may be a real-time operating system (RTOS) or program instructions in system firmware operating on the processor 310.

Program 380 may include stored instructions that direct the processor 310 to perform one or more steps toward processing signals received from substrate 102, as discussed above.

In some embodiments, the processor 310 may be configured to select the emitted set(s) of wavelengths and the respective distance(s) of the light source 108 from the first detectors 104, as discussed above. The input parameter may include, for example, a temperature, a lighting condition, a velocity, an acceleration, a change in acceleration, a pressure, a change in pressure, a volume, a change in volume, a measurement made, recorded, or calculated by the system, a communication from another device or system, or a combination thereof.

In some embodiments, the environmental sensor(s) 320 can measure parameters surrounding the patient and not the patient directly. Environmental properties may include, for example, temperature, humidity, pressure, motion, chemical composition, ambient light intensity, sound, etc., of the external environment in which the patient is positioned. For example, if the patient's body temperature is calculated as being too high, such as above some predetermined threshold, the electronics module 302, e.g., via the processor 310, may be configured to adjust a thermostat located in the room in which the patient is located. As another example, environmental sensor(s) 320 may include a microphone configured to receive spoken instructions informing the electronics module 302 how to operate.

In some embodiments, the electronics module 302 (e.g., 302A and/or 302B) may be configured to detect a configuration (e.g., a system architecture) of the substrate 102 when the electronics module 302 is communicatively connected to the substrate 102. Upon detecting the configuration of the substrate 102, electronics module 302 may be configured to adapt its own behavior to conform with the detected configuration. In some embodiments, electronics module 302 may include its own software and/or firmware, and upon being connected to the substrate 102, may be configured to determine whether the substrate 102 (e.g., with respect to its configuration) is compatible with the software and/or firmware. In some embodiments, if electronics module 302 determines that the substrate 102 is not compatible with the software and/or firmware, electronics module 302 may be configured to transmit an alert (e.g., to a computing device), and/or perform an update to its software and/or firmware such that the substrate 102 is compatible with the updated software and/or firmware. Electronics module 302 may be configured to update its software and/or firmware independently, e.g., via communicating internally with memory device 360, or dependently, e.g., via communicating with an external computing device to conduct an over-the-air (OTA) update.

In some embodiments, system 400 (FIG. 4) may include one or more components discussed herein, such as flexible material 202, substrate 102, one or more electronics modules 302 (e.g., 302A and/or 302B). In some embodiments, one or more of the components of system 400 may communicate via a network 402 such that, for example, electronics module 302 (e.g., 302A and/or 302B) may receive and process signals transmitted from substrate 102, as discussed herein.

In some embodiments, system 400 may be used to monitor multiple patients at one time (e.g., users who may or may not be geographically collocated). In some embodiments, the system may be configured to, for example, adjust a feedback device or environmental property, based on the different calculated biometric parameters across patients. In some embodiments, the biometric data calculated by the electronics module 302 (e.g., 302A and/or 302B) may be benefited by a patient other than the patient being monitored, for example, a patient who may be remote from the system.

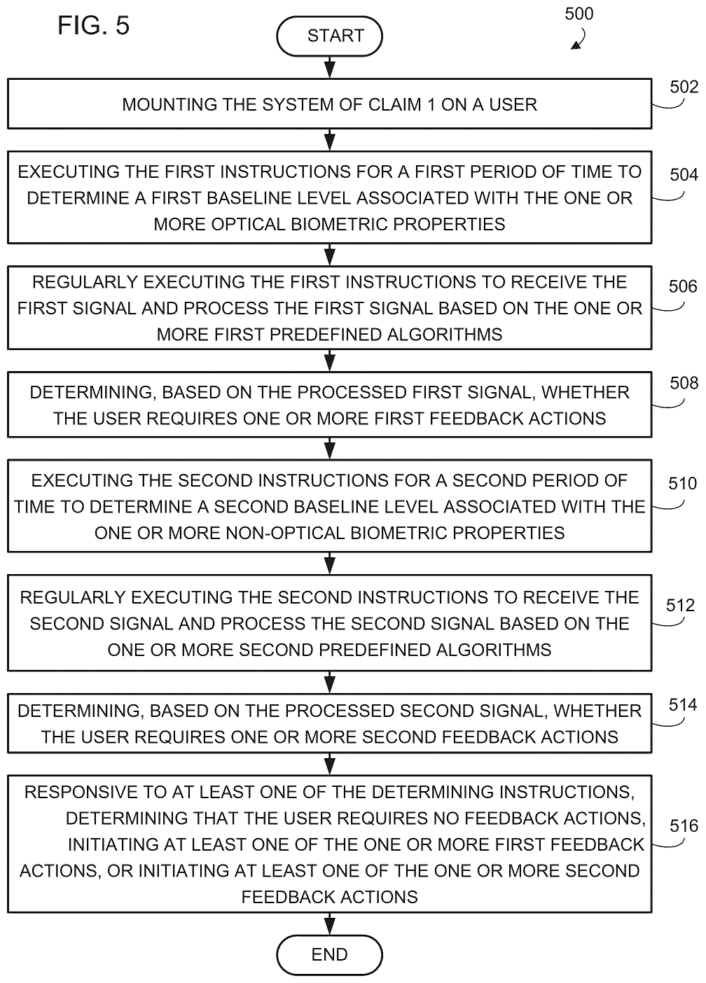

FIG. 5 is a flowchart of a method for detecting one or more biometric and neurophysiological parameters, in accordance with the present disclosure.

In block 502, the system 400 may be mounted on a user (e.g., on a part of the user's body, a piece of equipment and/or clothing worn by the user, etc.).

In block 504, the first instructions stored in the first memory device 360 of the electronics module 302A may be executed for a period of time (e.g., 2 minutes) to determine a baseline level associated with the one or more optical biometric parameters for the user.

In block 506, the first instructions may be regularly executed to receive the first signal and to process the first signal based on the one or more first predefined algorithms, as discussed herein.

In block 508, the first instructions may be further executed to determine, based on the processed first signal, whether the user requires one or more first feedback actions, as discussed herein.

In block 510, the second instructions stored in the second memory device 360 of the electronics module 302B may be executed for a period of time (e.g., 2 minutes) to determine a baseline level associated with the one or more non-optical biometric parameters for the user.

In block 512, the second instructions may be regularly executed to receive the second signal and to process the second signal based on the one or more second predefined algorithms, as discussed herein.

In block 514, the second instructions may be further executed to determine, based on the processed second signal, whether the user requires one or more second feedback actions, as discussed herein.

In block 516, responsive to at least one of the determining instructions, at least one of the first and second processors of the two respective electronics modules 302A, 302B may determine that the user requires no feedback actions, initiate at least one of the one or more first feedback actions, or initiate at least one of the one or more second feedback actions.

In some embodiments, the system 400 may integrate the functionality of electronics modules 302A and 302B into one electronics module 302 assimilating the hardware, functionality, and carrying out of the program instructions for both electronics modules within a single package.

In some embodiments, the system 400 may further include an external computing device including a memory and a computer processor. The external computing device may be connected to at least a portion of at least one of the processor and the memory device via a connection, wherein at least a portion of the program instructions is also stored on the external computing device.

The substrate 102 and/or the electronics module 302 can include a communication or connection interface 330. In some embodiments, the communication interface 330 can facilitate connections that can be, for example, a wireless connection (e.g., WiFi®), a wired connection, a Bluetooth connection, an NFC connection, a RFID connection, or a combination thereof. In some embodiments, data processing and real-time feedback may occur within the components onboard the substrate, or offboard through communication with the external computing device. The external computing device may comprise, for example, a smartphone, a charging or communications base station, a display screen, a tablet, a computer, a mobile or web-based application, or another device.

In some embodiments, the systems disclosed herein can be networked for concurrent monitoring of different physiological conditions of a user, the same or different physiological conditions at different locations on the body of a user, one or more physiological conditions of a group of wearers in a population, or a combination thereof.

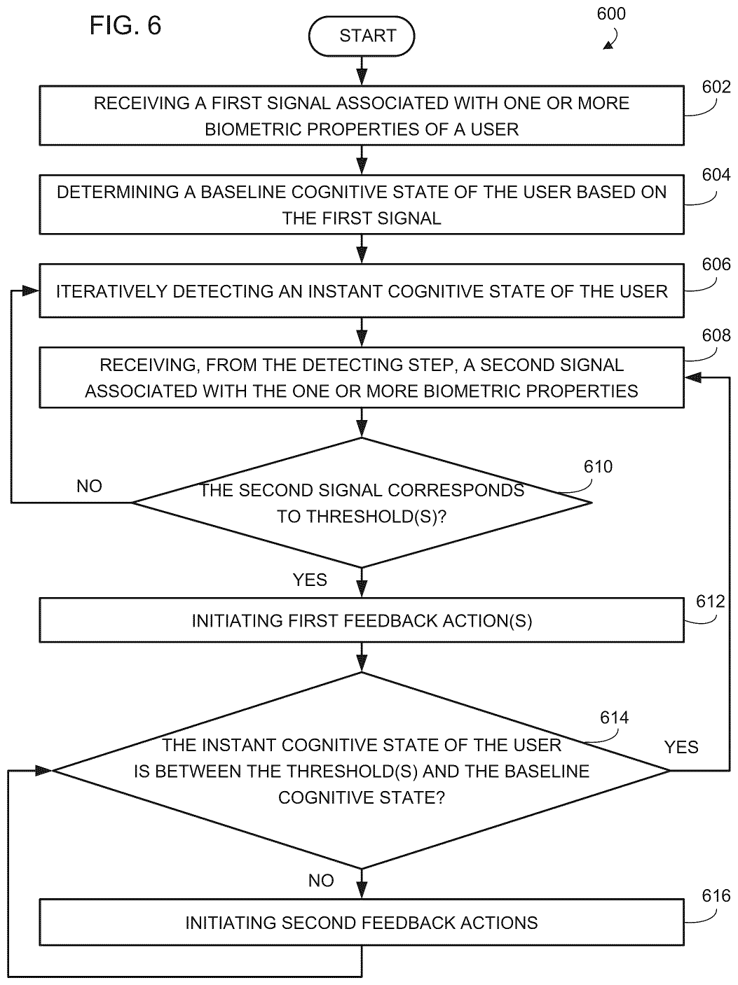

FIG. 6 is a flowchart of a method 600 for providing biofeedback to a user, in accordance with the present disclosure.

In block 602, one or more detectors may receive a first signal associated with one or more biometric properties of a user. The detector(s) may be those as discussed herein, such as detector(s) 104 and/or 106 of substrate 102. The biometric properties may include one or more optical biometric properties (e.g., detected via detector(s) 104) and one or more non-optical biometric properties (e.g., detected via detector(s) 106). As discussed herein, the optical biometric properties may include tissue oxygenation, heart rate, respiratory rate, oxygen saturation, blood pressure, and the like; the non-optical biometric properties may include EEG, EOG, EMG, bioimpedance circuitry, thermal properties, mechanical properties, and the like.

In block 604, one or more processors may determine a baseline cognitive and/or physiological state of the user based on the first signal. For example, processor(s) 310 of electronics module 302, as discussed herein, may process the first signal to determine a normal or resting state of the user before the user has begun any type of task (e.g., operating a vehicle, solving a puzzle, etc.).

In block 606, the detector(s) may iteratively detect an instant cognitive and/or physiological state of the user. For example, the detector(s) (e.g., detector(s) 104 and/or 106) may continuously detect a variety of optical and/or non-optical biometric properties of the user as the user performs one or more tasks, as discussed herein.

In block 608, the detector(s) may receive a second signal associated with the one or more biometric properties. For example, as the user works on a task, his/her cognitive and/or physiological state may change, such as by the user becoming fatigued, frustrated, stressed, etc. As such, the detector(s) may receive a second signal indicating a spike or fluctuation in the user's biometric properties, e.g., heart rate, blood pressure, etc.

In block 610, the processor(s) may determine whether the second signal corresponds to one or more thresholds. In some embodiments, the threshold(s) may be predetermined, e.g., based on the user's determined baseline state, as discussed above. In some embodiments, the threshold(s) may be fixed based on one or more factors, as further discussed below. In some embodiments, the system may be configured to adapt the threshold(s) as the user's cognitive state changes during the performance of a task, as further discussed herein. For example, the threshold(s) may correspond to the user's cognitive state at any point before the system determines whether to provide the user with feedback, as further discussed below.

In some embodiments, the threshold(s) may include a level of effort by the user in the task(s) he/she is performing, a level of engagement of the user in the task(s), a level of fatigue of the user in the task(s), and the like. The threshold(s) may be preconfigured based on the specific user being monitored. For example, a first user may be an expert in performing a certain task (e.g., flying a plane), and thus his/her level of effort, stress, frustration, etc., in performing such task may be much lower than a second user who has only performed that certain task once before. As such, the first user's threshold(s), for example, the points at which the first user's biometric properties may fluctuate or spike a certain distance above or below his/her baseline cognitive and/or physiological state, may be different from those points associated with the second user. In addition to being user-specific, the threshold(s) may also be task-specific as certain users may have an easier or more difficult time performing certain tasks, as discussed above.

In some embodiments, the processor(s) may determine that the second signal does not correspond to the threshold(s) of the user. For example, the processor(s) may determine that the user's biometric property readings, via the received signals, have not yet reached the point of this user's threshold(s), but are perhaps still closer to the user's initially determined baseline cognitive state such that the user does not yet require any type of feedback be provided, as further discussed below.

In block 612, responsive to the processor(s) determining that the second signal does correspond to the threshold(s) of the user, the processor(s) may initiate one or more first feedback actions. As discussed herein, the first feedback action(s) may include taking an additional optical biometric property reading, taking an additional non-optical biometric property reading, or providing one or more types of stimulation to the user. The type(s) of stimulation may be provided to the user, e.g., via the terminal(s) 110 on substrate 102, and may include tDCS, tACS, nVNS, TENS, TMS, sound stimulation (e.g., ultrasound, infrasound, binaural audio, etc.), optical stimulation, mechanical stimulation, and like.

In some embodiments, the first feedback action(s) may include a content modification, which may include, for example, a change in task type, a change in task difficulty, a change in training, a change in medical treatment, a delivery of one or more types of therapy, or a connection to or partnership with an Al system. These types of content modifications may be based on the system determining that the user requires some form of change in his/her current role, state, task, etc., to help bring the user's instant cognitive state back closer to his/her baseline cognitive state (e.g., the user may be performing a task that is making the user too stressed or fatigued).

In some embodiments, the user may be instructed (e.g., by a system manager) to change the type of task being conducted and/or the difficulty of the task. For example, the user may be instructed to stop working on solving a puzzle, and instead work on carrying objects from one location to another, and/or take a break from performing any task for a certain period of time. As another example, a system manager may change the difficulty level of a game the user is currently playing (e.g., a recreational game).

In some embodiments, the user's current type of training may be changed. For example, a system manager may provide an adjustment in the user's learning behavior during a virtual training, for example, by changing the pace of content delivery, the target(s) for learning and/or testing outcomes, etc.

In some embodiments, a change in medical treatment may be provided to the user. For example, a certain type and/or amount of medicine may be delivered to the user, e.g., pharmacologically or psychologically, based on the user's current cognitive state. For example, a user may have developed a disease or sickness which may have caused certain symptoms in the user, e.g., brain fog, pain, impacted mental health, etc. The system may thus be configured to deliver certain medical treatments to the user to help reduce the impact of these symptoms, and to continue to monitor the user, as discussed herein, to determine whether the provided medical treatment has provided a positive response from the user.

In some embodiments, certain types of therapy may be provided to the user. For example, the user may be provided with cognitive therapy, psychological therapy, physical therapy, or another kind of therapy that may positively impact the user's cognitive state.

In some embodiments, the user may be connected to or partnered with an AI system that may help to share the cognitive load of performing certain tasks with the user. For example, if the system determines the user may be stressed in performing a certain task, the user may be partnered with an AI system that can either share or take over the responsibility of performing such task such that the user's current cognitive state may come back closer to his/her baseline state. An AI system may provide a user with a semi-human interaction perspective toward short-term task effort (e.g., providing encouragement, focus, etc.).

In some embodiments, as discussed herein, the first feedback action(s) may be provided to the user via an environmental property, such as temperature, pressure, chemical composition, sound, light, motion, etc. For example, the system may be configured to transmit a signal to an external thermostat to increase or decrease the temperature in the user's environment based on the user's current body temperature.

In block 614, responsive to initiating the first feedback action(s), the processor(s) may determine whether the instant cognitive state of the user is between the threshold(s) and the user's baseline cognitive state. For example, the system may be configured to take additional biometric property readings of the user to determine whether the user's cognitive state has at least dropped below the user's predetermined threshold(s), but is still above the user's baseline cognitive state. Having the user's instant cognitive state be within this range may indicate to the system that the user is operating within an optimal range of stimulation in relation to the type and difficulty level of task the user is performing.

In some embodiments, when the system determines the user's instant cognitive state is between the predetermined threshold(s) and the user's baseline cognitive state, the system may be configured to re-start its feedback loop, e.g., to continue to monitor the user's instant cognitive state by taking continuous readings of the user's biometric properties until the detector(s) receive the next second signal associated with the user's biometric properties, as discussed above in block 608.

In block 616, responsive to the system determining that the user's instant cognitive state is not between the predetermined threshold(s) and the user's baseline cognitive state, for example, meaning the user may still be in a state of stress, fatigue, frustration, low engagement, etc., the system may be configured to initiate, e.g., via the processor(s), one or more second feedback actions. In some embodiments, the second feedback action(s) may be selected from the same grouping of feedback action(s) as discussed above with respect to the first feedback action(s). The goal of selecting the second feedback action(s) is to provide adaptive feedback to the user based on the user's instant cognitive state and to then once again continuously monitor the user's instant cognitive state to determine whether it has now fallen within the range of the threshold(s) and the user's baseline cognitive state, as discussed above in block 614.

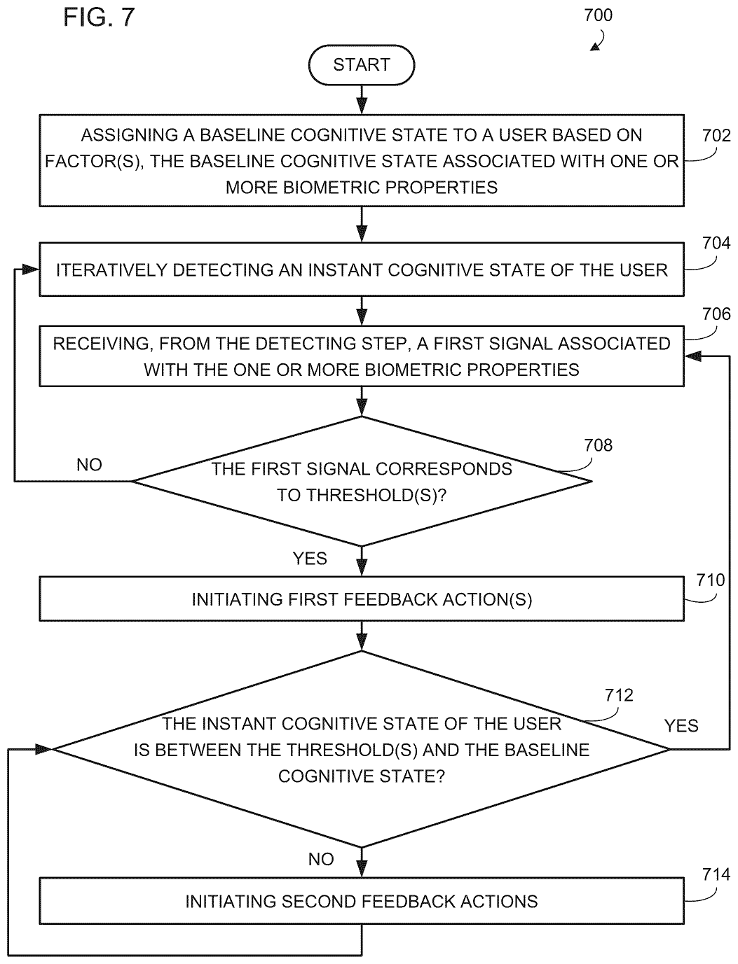

FIG. 7 is a flowchart of a method 700 for providing biofeedback to a user, in accordance with the present disclosure. Method 700 is similar to method 600 of FIG. 6, except that method 700 involves assigning a default baseline cognitive state to a user instead of determining the baseline cognitive state. The respective descriptions of blocks 704, 706, 708, 710, 712, and 714 of method 700 may be the same as or similar to the respective descriptions of blocks 606, 608, 610, 612, 614, and 616 of method 600 and as such, are not repeated herein for brevity.

In block 702, one or more processors (e.g., processor(s) 310 of electronics module 302) may assign a baseline cognitive state to the user based on one or more factors, where the baseline cognitive state is associated with one or more biometric properties (as discussed herein). In some situations, for example, the system may not have had an opportunity to determine the user's baseline cognitive state, as discussed above in block 604 of method 600. For example, a user may become ill with a sickness or disease. The systems and methods disclosed herein may thus be used to monitor the user's cognitive state to help the user recover from such sickness or disease. In such example, the system may not have first had an opportunity to determine the user's baseline cognitive state prior to the user contracting the sickness or disease.

In some embodiments, the one or more factors upon which the user's default baseline cognitive state may be based, may include age, sex, gender, education level, profession, medical history, content familiarity, task engagement, task effort, task performance, environmental factors (e.g., that may affect the user), and the like. For example, a user may be a 45 year old male, with a college education and no significant historical health issues. The system may be configured to assign the user a baseline cognitive state, or an assumed state of where the user's various biometric properties may tend to be when in a resting state, based on these personal factors. The system may then use this default baseline cognitive state in monitoring and providing adaptive feedback to the user, as discussed herein.

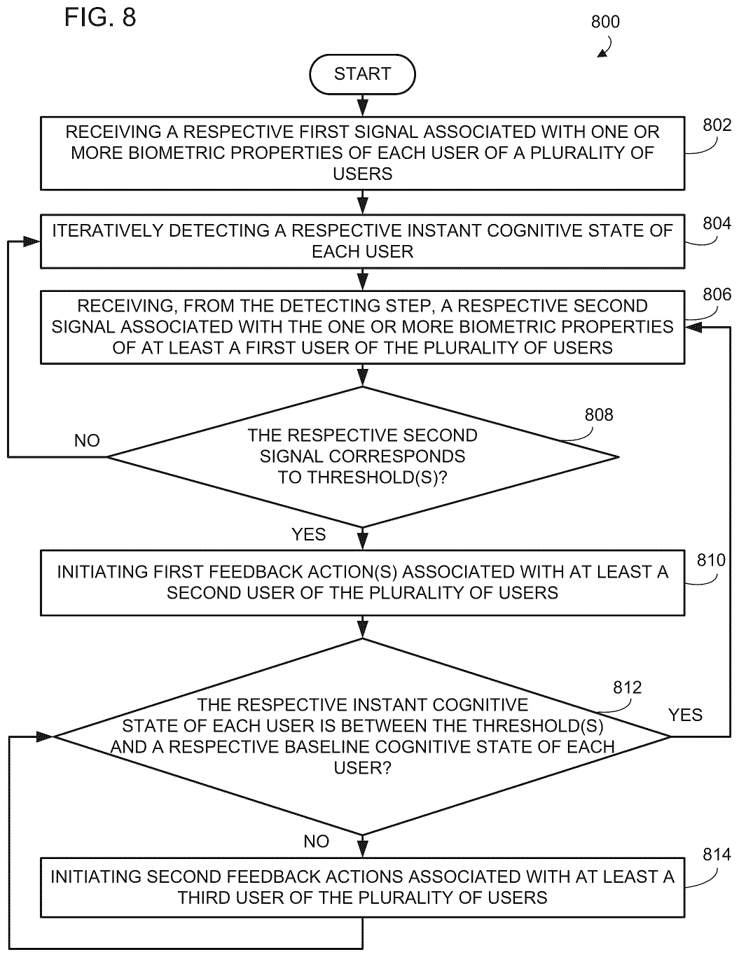

FIG. 8 is a flowchart of a method 800 for providing biofeedback to a plurality of users, in accordance with the present disclosure. Method 800 is similar to methods 600 and 700 of FIGS. 6 and 7, respectively, except that method 800 involves monitoring the respective cognitive state of multiple users simultaneously and providing adaptive biofeedback to the users to enhance group or team cognitive function.

In block 802, one or more detectors (e.g., detector(s) 104 and/or 106 of substrate 102) may receive a first signal associated with one or more biometric properties of each user of a plurality of users. This step may be similar to block 602 of method 600, except that this step involves receiving a first signal unique to each user of a group of users.

In block 804, the detector(s) may iteratively detect a respective instant cognitive state of each user of the plurality of users. This step may be similar to block 606 of method 600, except that this step may involve continuously detecting a variety of optical and/or non-optical biometric properties of each user of a group of users.

In block 806, the detector(s) may receive a second signal associated with the one or more biometric properties of at least a first user of the plurality of users. For example, as the system continuously monitors each user simultaneously, the system may not receive a second signal, as discussed herein, associated with all members of the plurality, but instead may receive the second signal corresponding to one or more of the group members.

In block 808, the processor(s) may determine whether the second signal corresponds to one or more thresholds. This step may be the same as or similar to block 610 of method 600, as discussed above.

In block 810, responsive to determining the respective second signal corresponds to the threshold(s), the processor(s) may initiate one or more first feedback actions (as discussed herein) associated with at least a second user of the plurality of users. In some embodiments, the second user may be the same user or a different user from the first user, as discussed above in block 806. For example, the system may be configured, as it continuously monitors the cognitive state of all members of a group, to compare the instant cognitive states of each user to determine which user(s) may require first feedback action(s). For example, if the system received the first signal (block 806) of a first user, and determined the first signal to indicate the first user is over-stressed in performing a certain task, the system may provide a first feedback action to a different, second user, for example, to instruct the second user to assist the first user in completing the task. Alternatively, or in addition, the system may be configured to provide a first feedback action to the first user, such as instructing the first user to take a break from performing the current task, or to switch to performing a different task to help bring the first user's stress level back down closer to the first user's baseline cognitive state.

In block 812, after providing at least the second user with first feedback action(s), as discussed above, the processor(s) may determine whether the respective instant cognitive state of each user is between the threshold(s) and a respective baseline cognitive state of each user. This step may be similar to block 614 of method 600, except that this step involves making such determination for each of the members of the group. In some embodiments, the respective baseline cognitive state of each user may be a determined baseline cognitive state, as discussed above in block 604 of method 600, or an assigned baseline cognitive state, as discussed above in block 702 of method 700. In some embodiments, the system may be configured to determine a respective baseline cognitive state for some of the users of a group, while assigning a respective baseline cognitive state for other users of the group. In some embodiments, the system may be configured to evaluate each individual user's instant cognitive state compared to such user's baseline cognitive state to determine whether one or more users of the group may require additional feedback action(s), as discussed herein.

In some embodiments, when the system determines each user's respective instant cognitive state is between the threshold(s) and each user's respective baseline cognitive state, the system may be configured to re-start its feedback loop, e.g., to continue to monitor each user's instant cognitive state by taking continuous readings of each user's biometric properties until the detector(s) receive the next second signal associated with at least a first user of the plurality of users, as discussed above in block 806.

In block 814, responsive to the system determining that each user's respective instant cognitive state is not between the threshold(s) and each user's respective baseline cognitive state, for example, meaning at least one member of the group may still be in a state of stress, fatigue, frustration, low engagement, etc., the system may be configured to initiate, e.g., via the processor(s), one or more second feedback actions associated with at least a third user of the plurality of users. This step may be similar to block 616 of method 600, except that in this step, the system may be configured to provide second feedback action(s) to one or multiple members of the group. In some embodiments, the third user may be the same user or a different user from the first and second users, as discussed above with respect to blocks 806 and 810, respectively.

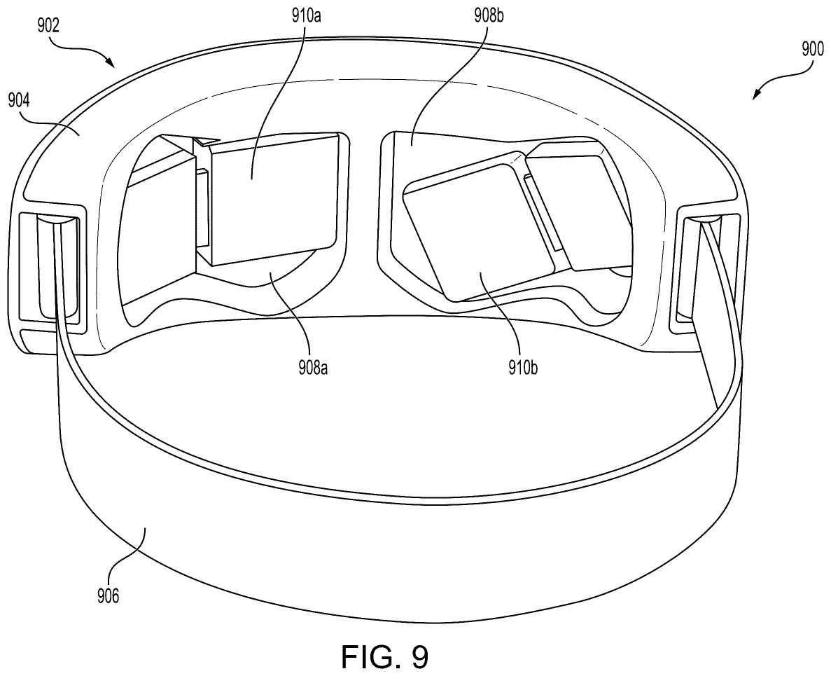

FIG. 9 depicts a system 900 that can be used for detecting biometric and/or neurophysiological parameters of a user. The system 900 can include a wearable device 902, such as a visor or headset, that can be worn on the user's body, such as on the user's head (e.g., forehead). The wearable device 902 can include a first portion 904 configured to fit across a user's forehead, and an adjustable strap 906 used to tighten or loosen the wearable device 902 such that it fits properly on the user's head. The wearable device 902 can also include one or more cutouts or slots 908 (e.g., 908a, 908b) configured to house one or more components of the system 900, such as one or more substrates 910 (e.g., 910a, 910b). In such embodiments, the substrate(s) 910 may include one or more of the same or similar features as substrate 102, as discussed above. However, substrate(s) 910 may be configured such that it can fit and/or lock into the slot(s) 908. In this way, substrate(s) 910 can aid in the system 900 being a modular system. For example, a first substrate 910a (e.g., monitoring one or more optical biometric properties of the user) may be integrated into the wearable device 902 (e.g., via slot 908a), while a second substrate 910b (e.g., monitoring one or more non-optical biometric properties of the user) may be attached to another part of the user's body, such as the user's chest. In this way, the system 900 can be configured to monitor or detect biometric and/or neurophysiological parameters of the user from one or more of the user's body parts.

System 900 can also include one or more electronics modules 302, as discussed above, that can themselves be integrated into the wearable device 902 and/or be disposed external to the wearable device 902 (e.g., via a wired or wireless connection). The electronics module(s) 302 of the system 900 can also be configured in a modular fashion including, for example, one or more electronics modules 302 integrated into the wearable device 902 (e.g., internal to a substrate 910, or external to the substrate 910 but still integrated into the wearable device 902 via another slot 908), and/or one or more electronics modules 302 that are external to the wearable device 902 (e.g., via a wired or wireless connection).

System 900 can also include one or more components to support the wearable device 902 being a remote or wireless device. For example, wearable device 902 can include a rechargeable battery. In some embodiments, the wearable device 902 may include one or more internal processors that may be integrated into the wearable device 902 but are separate from the electronics module(s) 902. These internal processor(s) may include one or more of the same or similar features as electronics module(s) 302, as discussed herein.

Although some of the processing systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc.

In some embodiments, the systems and methods described herein include independent wireless devices communicating biometrics information about different areas of tissue (e.g., the brain) simultaneously. In some embodiments, the systems and methods described herein include scanning a single device over different areas of the body and continuously imaging tissue, changing methods based on determined tissue state or changes in patient condition.

In some embodiments, the systems and methods described herein include two or more independent systems that can simultaneously interrogate multiple areas of cerebral and somatic tissue to interrelate physiological status (for example, tissue oxygenation) in each area. These areas may have significantly different oxygenation signatures at any given time and simultaneously sampling these is particularly important to understand situations of local or central fatigue or recovery onset by the user. Simultaneous imaging of different body systems can also elucidate generalized physiological condition, for instance indicating systemic response to exogenous conditions such as carbon monoxide poisoning or endogenous conditions such as hemorrhage. The independently sampled processed data from each area of the body may then send signals to a user interface if a specific tissue level, condition, or status is reached, or stream data to the external processing module for real-time interpretation, or both.

In some embodiments, the functional near-infrared spectroscopy systems and methods described herein include independent wireless devices communicating multi-point physiological information (e.g., oxygenation) about the brain and body simultaneously.

In some embodiments, the systems and methods described herein include multiple systems that can be worn by multiple different individuals whose data is integrated to form a comprehensive image of a group of individuals' health. This integration can be simultaneous for co-located users or asynchronous for disparate groups, or another combination. For example, comparing real-time physiological monitoring across multiple individuals can enable population monitoring and a more holistic image of group performance and wellness. Such continuous imaging can identify early threats or enhancements and increase risk or opportunity for better group performance and outcome.

As an example, a set of n systems 400 may be placed on the heads or bodies of n users. Each system is as described herein and includes an LED user interface light indicating a green/yellow/red indication of tissue health. Based on individual physiology of the n users, the n systems 400 monitor users in this cohort depending on the individual user's needs. In an example, one system in the user cohort begins sending abnormal signals back to the external processing unit indicating the onset of cognitive or physiological change in the target user that may have implications for the state of the rest of the user cohort, providing earlier notification from earlier surveillance and allowing real-time adaptation to monitored changes in condition.

In some embodiments, the systems and methods described herein include monitoring population health through a network of individual users' biometric detection systems. In some embodiments, this can enable broader decision making and earlier insight into performance degradations or risks from proximity to decompensating near neighbors. For example, the monitored conditions can include pre-symptomatic detection of infection, fatigue, or environmental exposure and the implementation of remedial strategies to optimize outcomes.

Where any component discussed herein is implemented in the form of firmware and/or software, any one of a number of programming languages may be employed such as, for example, C, C++, C#, Objective C, Java®, JavaScript®, Perl, PHP, Visual Basic®, Python®, Ruby, Flash®, or other programming languages. A number of firmware and/or software components are stored in the memory and are executable by the processor. In this respect, the term “executable” means a program file that is in a form that can ultimately be run by the processor. Examples of executable programs can be, for example, a compiled program that can be translated into machine code in a format that can be loaded into a random access portion of the memory and run by the processor, source code that can be expressed in proper format such as object code that is capable of being loaded into a random access portion of the memory and executed by the processor, or source code that can be interpreted by another executable program to generate instructions in a random access portion of the memory to be executed by the processor, etc. An executable program can be stored in any portion or component of the memory including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory is defined herein as including both volatile and nonvolatile memory and data storage components. Volatile components are those that do not retain data values upon loss of power. Nonvolatile components are those that retain data upon a loss of power. Thus, the memory can include, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM can include, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor can represent multiple processors and/or multiple processor cores and the memory can represent multiple memories that operate in parallel processing circuits, respectively. In such a case, the local interface can be an appropriate network that facilitates communication between any two of the multiple processors, between any processor and any of the memories, or between any two of the memories, etc. The local interface can include additional systems designed to coordinate this communication, including, for example, performing load balancing. The processor can be of electrical or of some other available construction.

Although some of the processing systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc.

It should be understood that any logic or application described herein that incorporates software or code can be embodied in any non-transitory computer-readable medium for use by or in connection with an instruction execution system such as, for example, a processor in a computer system or other system. In this sense, the logic can include, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. The computer-readable medium can incorporate any one of many physical media such as, for example, magnetic, optical, or semiconductor media. More specific examples of a suitable computer-readable medium include, but are not limited to, magnetic tapes, magnetic floppy diskettes, magnetic hard drives, memory cards, solid-state drives, USB flash drives, or optical discs. Also, the computer-readable medium can be a random access memory (RAM) including, for example, static random access memory (SRAM) and dynamic random access memory (DRAM), or magnetic random access memory (MRAM). In addition, the computer-readable medium can be a read-only memory (ROM), a programmable read-only memory (PROM), an erasable programmable read-only memory (EPROM), an electrically erasable programmable read-only memory (EEPROM), or other type of memory device.

Further, any logic or application described herein can be implemented and structured in a variety of ways. For example, one or more applications described can be implemented as modules or components of a single application. Further, one or more applications described herein can be executed in shared or separate computing devices or a combination thereof. For example, a plurality of the applications described herein can execute in the same computing device 515, or in multiple computing devices in the same computing environment. Additionally, it is understood that terms such as “application,” “service,” “system,” “engine,” “module,” and so on may be interchangeable and are not intended to be limiting.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A method for providing biofeedback to a user, the method comprising:
receiving, by one or more detectors, a first signal associated with one or more biometric properties of the user;
determining, by one or more processors, a baseline cognitive state of the user based on the first signal;
iteratively detecting, by the one or more detectors, an instant cognitive state of the user;
receiving, from the detecting step, a second signal associated with the one or more biometric properties;
determining, by the one or more processors, whether the second signal corresponds to one or more thresholds;
responsive to determining that the second signal corresponds to the one or more thresholds, initiating, by the one or more processors, one or more first feedback actions;
responsive to initiating the one or more first feedback actions, determining whether the instant cognitive state of the user is between the one or more thresholds and the baseline cognitive state; and
responsive to determining the instant cognitive state of the user is not between the one or more thresholds and the baseline cognitive state, initiating, by the one or more processors, one or more second feedback actions.

2. The method of claim 1, wherein the one or more biometric properties comprise one or more optical biometric properties and one or more non-optical biometric properties.

3. The method of claim 2, wherein:
the one or more optical biometric properties comprise one or more of tissue oxygenation, heart rate, respiratory rate, oxygen saturation, blood pressure, or combinations thereof; and
the one or more non-optical biometric properties comprise one or more of electroencephalography (EEG), electrooculography (EOG), electromyography (EMG), bioimpedance circuitry, thermal properties, mechanical properties, or combinations thereof.

4. The method of claim 1, wherein the one or more thresholds are based on a level of effort by the user in one or more tasks, a level of engagement of the user in the one or more tasks, a level of fatigue of the user in the one or more tasks, or combinations thereof.

5. The method of claim 1, wherein the one or more first and second feedback actions comprise one or more of an optical biometric property reading, a non-optical biometric property reading, one or more types of stimulation, a content modification, or combinations thereof.

6. The method of claim 5, wherein:
the one or more types of stimulation comprise one or more of transcranial direct current stimulation (tDCS), transcranial alternating current stimulation (tACS), vagus nerve stimulation (nVNS), transcutaneous electrical nerve stimulation (TENS), transcranial magnetic stimulation (TMS), sound stimulation, optical stimulation, mechanical stimulation, or combinations thereof.

7. The method of claim 5, wherein the content modification comprises one or more of a change in task type, a change in task difficulty, a change in training, a change in medical treatment, a delivery of one or more types of therapy, a connection to an artificial intelligence (AI) system, or combinations thereof.

8. The method of claim 7, wherein the one or more types of therapy comprise one or more of cognitive therapy, psychological therapy, physical therapy, or combinations thereof.

9. The method of claim 5, wherein the one or more first and second feedback actions are provided to the user via an environmental property comprising one or more of temperature, pressure, chemical composition, sound, light, motion, or combinations thereof.

10. A method for providing biofeedback to a user, the method comprising:
assigning, by one or more processors, a baseline cognitive state to the user based on one or more factors, the baseline cognitive state associated with one or more biometric properties;
iteratively detecting, by one or more detectors, an instant cognitive state of the user;
receiving, from the detecting step, a first signal associated with the one or more biometric properties;
determining, by the one or more processors, whether the first signal corresponds to one or more thresholds;
responsive to determining that the first signal corresponds to the one or more thresholds, initiating, by the one or more processors, one or more first feedback actions;
responsive to initiating the one or more first feedback actions, determining whether the instant cognitive state of the user is between the one or more thresholds and the baseline cognitive state; and
responsive to determining the instant cognitive state of the user is not between the one or more thresholds and the baseline cognitive state, initiating, by the one or more processors, one or more second feedback actions.

11. The method of claim 10, wherein the one or more factors comprise one or more of age, sex, gender, education level, profession, medical history, content familiarity, task engagement, task effort, task performance, environmental factors, or combinations thereof.

12. The method of claim 10, wherein:
the one or more biometric properties comprise one or more optical biometric properties and one or more non-optical biometric properties;
the one or more optical biometric properties comprise one or more of tissue oxygenation, heart rate, respiratory rate, oxygen saturation, blood pressure, or combinations thereof; and
the one or more non-optical biometric properties comprise one or more of electroencephalography (EEG), electrooculography (EOG), electromyography (EMG), bioimpedance circuitry, thermal properties, mechanical properties, or combinations thereof.

13. The method of claim 10, wherein the one or more thresholds are based on one or more of a level of effort by the user in one or more tasks, a level of engagement of the user in the one or more tasks, a level of fatigue of the user in the one or more tasks, or combinations thereof.

14. The method of claim 10, wherein the one or more first and second feedback actions comprise one or more of an optical biometric property reading, a non-optical biometric property reading, one or more types of stimulation, a content modification, or combinations thereof.

15. The method of claim 14, wherein the content modification comprises one or more of a change in task type, a change in task difficulty, a change in virtual training, a change in medical treatment, a delivery of one or more types of therapy, a connection to an artificial intelligence (AI) system, or combinations thereof.

16. A method for providing biofeedback to a plurality of users, the method comprising:
receiving, by one or more detectors, a respective first signal associated with one or more biometric properties of each user of the plurality of users;
iteratively detecting, by the one or more detectors, a respective instant cognitive state of each user;
receiving, from the detecting step, a respective second signal associated with the one or more biometric properties of at least a first user of the plurality of users;
determining, by the one or more processors, whether the respective second signal corresponds to one or more thresholds;
responsive to determining that the respective second signal corresponds to the one or more thresholds, initiating, by the one or more processors, one or more first feedback actions associated with at least a second user of the plurality of users;
responsive to initiating the one or more first feedback actions, determining whether the respective instant cognitive state of each user is between the one or more thresholds and a respective baseline cognitive state of each user; and
responsive to determining the respective instant cognitive state of each user is not between the one or more thresholds and the respective baseline cognitive state, initiating, by the one or more processors, one or more second feedback actions associated with at least a third user of the plurality of users.

17. The method of claim 16, wherein:
the one or more biometric properties comprise one or more optical biometric properties and one or more non-optical biometric properties;
the one or more optical biometric properties comprise one or more of tissue oxygenation, heart rate, respiratory rate, oxygen saturation, blood pressure, or combinations thereof; and
the one or more non-optical biometric properties comprise one or more of electroencephalography (EEG), electrooculography (EOG), electromyography (EMG), bioimpedance circuitry, thermal properties, mechanical properties, or combinations thereof.

18. The method of claim 16, wherein the one or more thresholds are based on one or more of a level of effort by each user in one or more tasks, a level of engagement of each user in the one or more tasks, a level of fatigue of each user in the one or more tasks, or combinations thereof.

19. The method of claim 16, wherein the one or more first and second feedback actions comprise one or more of an optical biometric property reading, a non-optical biometric property reading, one or more types of stimulation, a content modification, or combinations thereof.

20. The method of claim 16, wherein:
at least two of the first, second, and third users are the same.
